(19) United States
(12) Patent Application Publication
(10) Pub. No.: US 2026/0224799 A1
(43) Pub. Date: Aug. 6, 2026

(54) Method, device and procedure for constipation relief without the use of drugs and medications, or adjustment to the lifestyle and food consumption.

(71) Applicant: Leonid Frid, Mountain View, CA (US)

(72) Inventor: Leonid Frid, Mountain View, CA (US)

(21) Appl. No.: 18/944,746

(22) Filed: Nov. 12, 2024

(51) Int. Cl. A61M 3/02

(52) U.S. Cl. CPC A61M 3/0279; A61M 3/0204; A61M 2205/3368; A61M 2210/1067

(57) ABSTRACT

A method, device, and procedure for inducing bowel movements without the use of drugs, lifestyle changes, or dietary adjustments. The invention utilizes a temperature-controlled medium directed at the anus of a patient to induce involuntary rectal reflux, prompting a bowel movement. The medium—such as water—is heated to a temperature range of between 40° C. to 55° C., depending on patient sensitivity, to trigger rectal wall stretching and subsequent rectal reflux, facilitating bowel movement and waste excretion. Beyond promoting regular bowel function and constipation relief, this method aids lipid metabolism by promoting waste removal, modulating gut microbiota, and regulating bile acid levels and liver cholesterol uptake. These effects offer potential benefits in managing cholesterol and triglyceride levels. The method is suitable for personalized and clinical applications and can be implemented with devices adaptable for both individual and therapeutic use.

### 2. BACKGROUND OF THE INVENTION

Chronic constipation is a widespread health concern affecting millions worldwide. It is one of the most common gastrointestinal disorders, with an estimated global prevalence ranging from 10% to 20%. Bowel movements result from a complex coordination of muscle contractions, nerve signals, and hormonal responses that ensure the digestion of food, absorption of nutrients, and elimination of waste.

### Key Aspects of Chronic Constipation

Global Prevalence: Approximately 14% of the global population experiences chronic constipation, with rates varying from 10% to 20% in Western countries.

U.S. and Developed Countries: In the United States, around 15% of the population, or roughly 45 million individuals, suffer from chronic constipation, with women and the elderly disproportionately affected.

Health and Economic Impact: Chronic constipation can significantly affect quality of life, leading to discomfort, pain, and complications such as hemorrhoids or anal fissures. It also imposes a substantial economic burden, contributing to healthcare costs through medical consultations, diagnostic tests, medications, and missed workdays. In the U.S., direct costs exceed $230 million annually.

Risk Factors: Factors such as aging, gender (with women being more susceptible), low fiber intake, physical inactivity, and certain medical conditions (e.g., diabetes, Parkinson's disease) and medications (e.g., opioids) can increase the risk of constipation.

Public Awareness: Despite its prevalence, many people do not seek medical help due to embarrassment or the belief that constipation is not a medical issue, leading to underreporting.

Importance of Bowel Movements for Overall Health: Regular bowel movements are essential to maintaining health by removing waste and toxins from the body. They play a crucial role in the digestive process, which involves extracting nutrients, water, and energy from food, and expelling waste materials that are not useful to the body. This process helps maintain a clean digestive tract and prevents the accumulation of harmful bacteria and toxins.

### Bowel Movements Regularity Benefit Health By:

Eliminating Waste and Toxins: Bowel movements expel unnecessary substances such as dead cells, bacteria, and undigested food particles, supporting the body's natural detoxification process.

Promoting Digestive Health: Consistent, healthy bowel movements indicate a functioning digestive system, which is essential for nutrient absorption and energy production.

Preventing Infections and Diseases: Efficient waste elimination reduces the risk of gastrointestinal conditions, such as diverticulitis and colorectal cancer.

Maintaining Hydration and Electrolyte Balance: The colon reabsorbs water and electrolytes from stool, maintaining hydration and electrolyte levels essential for bodily functions.

Signaling Health Issues: Changes in stool frequency, color, or consistency can indicate dehydration, infections, irritable bowel syndrome (IBS), or dietary problems.

Mechanism of Bowel Movement: The process of bowel movement, or the expulsion of waste from the digestive system, involves coordinated actions across multiple structures, including muscles, nerves, and hormones. The following summarizes this process:

### Mouth and Esophagus:

Ingestion and Chewing: Food is chewed and mixed with saliva to form a bolus.

Swallowing and Propulsion: The bolus moves through the esophagus via peristalsis, a series of wave-like muscle contractions.

### Stomach and Small Intestine:

Stomach Digestion: Digestive enzymes and acids in the stomach break down food into a semi-liquid mixture called chyme.

Small Intestine Absorption: In the small intestine, most nutrient absorption occurs. Peristalsis helps push chyme along, while villi absorb nutrients into the bloodstream.

### Large Intestine (Colon):

Water Absorption: The colon absorbs water and electrolytes, forming solid waste.

Microbial Fermentation: Beneficial bacteria ferment undigested carbohydrates, producing short-chain fatty acids and gases.

Formation of Feces: As water is absorbed, waste consolidates into feces, which is slowly pushed toward the rectum.

### Peristalsis and Segmentation Movements:

Peristalsis: Coordinated muscle contractions propel food and waste throughout the digestive tract.

Segmentation: Segmental movements help mix contents for better absorption.

### Defecation Reflex:

Rectum and Anus Filling: Feces moving into the rectum trigger stretch receptors that signal the brain.

Internal and External Anal Sphincters: These sphincters allow for voluntary and involuntary control over defecation.

### Hormonal and Neural Regulation:

Enteric Nervous System (ENS): The ENS coordinates muscle contractions and glandular secretions.

Autonomic Nervous System: The parasympathetic nervous system stimulates digestive activity, while the sympathetic nervous system inhibits it.

Hormones: Hormones like gastrin and motilin regulate digestive secretions and bowel movement speed.

### Influence of Diet, Hydration, and Lifestyle:

Fiber: Fiber adds bulk to stool, aiding movement through the intestines.

Hydration: Adequate water intake prevents constipation.

Physical Activity: Exercise can stimulate the digestive tract and promote regular bowel movements.

Metabolic and Health Implications of Bowel Movements:

Bowel movements are essential for metabolic health, supporting nutrient absorption, maintaining gut microbiome balance, eliminating waste, and regulating hormones.

Known Methods to Stimulate Bowel Movements: Various known methods and treatments for constipation include dietary adjustments, physical exercise, biofeedback, and medication. However, these methods generally require either lifestyle modifications or medications.

### 3. SUMMARY OF THE INVENTION

The present invention proposes a new method for stimulating bowel movements without drugs or lifestyle changes, addressing an unmet need in current constipation treatments.

This invention utilizes the discovery of an involuntary rectal reflex triggered by delivering a temperature-controlled medium to the patient's anus.

Devices with temperature-regulating capabilities for delivering a directed flow of heated medium can vary in design, but they must be capable of providing a regulated temperature to the patient's anus. The specific temperature used is based on the patient's individual skin sensitivity.

This method addresses the issue of bowel movement regularity and provides constipation relief, allowing patients to induce bowel movements as needed.

The proposed method also has secondary applications in influencing lipid metabolism through waste removal. This process can modulate gut microbiota, regulate bile acid levels, and impact nutrient processing within the gastrointestinal tract.

Additionally, the invention promotes enhanced metabolic efficiency by supporting regular bowel function and potentially offers therapeutic benefits for managing cholesterol, triglycerides, and related lipid disorders.

The method is adaptable for both personalized and clinical applications.

This invention claims protection for all applications and variations that utilize the disclosed method, as well as for any potential device designs capable of delivering temperature-controlled mediums for the purposes of regulating bowel movements and influencing lipid metabolism.

### 4. DETAILED DESCRIPTION OF THE INVENTION AND PROCEDURE

### Invention Description:

The invention utilizes the discovery of an involuntary rectum reflex, triggered by delivering a temperature-controlled medium to the anus of a patient.

Devices designed for this procedure, such as bidets, can vary widely. These devices are intended to deliver a directed flow of temperature-controlled mediums, such as water, to the anus. The temperature range for the medium should be between 40° C. and 55° C., adjusted according to the patient's individual skin sensitivity.

The process involves anus relaxation, stretching the rectal walls, which activates stretch receptors that signal the brain.

This activation results in involuntary muscle relaxation in response to rectal distention. Coupled with internal anal sphincter relaxation, this triggers the urge to defecate.

The external anal sphincter, which is under voluntary control, allows the person to initiate waste release at will.

This method provides a reliable approach to bowel movement stimulation, allowing patients to induce bowel movements as needed.

In addition to aiding bowel movement, this method may influence lipid metabolism by promoting waste removal. This waste elimination can positively impact gut microbiota, bile acid regulation, and nutrient processing within the gastrointestinal tract.

The invention may also enhance metabolic efficiency by supporting regular bowel function, with potential therapeutic benefits for managing cholesterol, triglycerides, and related lipid disorders. Preliminary tests have shown the following changes after one week of use: a 21% reduction in triglycerides, a 23% reduction in VLDL cholesterol, a 14.8% reduction in Apolipoprotein B, and a 19.1% increase in HDL cholesterol.

The method is suitable for both personalized and clinical use.

This invention claims protection for all applications and variations utilizing the disclosed method, as well as for all potential device designs that deliver temperature-controlled mediums to the anus for regulating bowel movements and influencing lipid metabolism in humans and potentially animal subjects.

### Procedure:

The patient sits on the toilet equipped with a device for delivery of the temperature controlled heated medium.

The patient activates the medium flow and begins to gradually increase the temperature.

At first, the medium gently massages the anus, helping the patient become accustomed to the sensation and temperature.

As the temperature rises, the sensation intensifies. When the medium becomes noticeably warm, the patient will begin to feel an urge for bowel movement.

When the medium temperature reaches in between 40° C. and 55° C., according to the patient's individual skin sensitivity, the rectum reflexively responds, leading to bowel movement initiated by repeated contractions.

The bowel movement typically lasts 5-15 seconds.

Waste is excreted into the toilet bowl.

Afterward, the medium temperature should be reduced to a comfortable level for cleaning, around 35° C.-40° C., to complete the process.

The procedure concludes with a final wash, completing the waste elimination and cleansing process.

A method for inducing bowel movement without pharmacological agents or lifestyle modifications, comprising:
Delivering a temperature-controlled medium to the anus of a patient;
Adjusting the temperature of the medium to induce involuntary rectal reflux and stimulate the urge to defecate;
Wherein the temperature of the medium is set within a range of between 40° C. to 55° C., based on the patient's individual sensitivity.

2. The method of claim 1, further comprising:
A step for regulating lipid metabolism by promoting bowel regularity, thereby modulating gut microbiota, bile acid levels, and nutrient processing within the gastrointestinal tract.
A device for delivering a temperature-controlled medium to stimulate bowel movement, comprising:
A system for directing a controlled flow of heated medium to the anus;
A temperature control feature allowing adjustments between 40° C. and 55° C.;
Wherein the device enables the patient to initiate bowel movement by activating rectal reflux.

3. The device of claim 3, further comprising:
A bidet-style nozzle configured for direct, controlled application of the heated medium to the anus.

4. The method of claim 1, wherein:
The temperature-controlled medium causes stretching of the rectal walls, activating stretch receptors, which signal the brain to initiate defecation.
